(19) United States
(12) Patent Application Publication
(10) Pub. No.: US 2004/0197286 A1
(43) Pub. Date: Oct. 7, 2004

(54) Cosmetic composition comprising the combination of a specific ester and silicone compound

(75) Inventors: Valerie Robert, Paris (FR); Pascal Arnaud, L'Hay Les Roses (FR)

Correspondence Address: FINNEGAN, HENDERSON, FARABOW, GARRETT & DUNNER, LLP, 1300 I STREET, NW, WASHINGTON, DC, 20005, US

(73) Assignee: L'OREAL S.A.

(21) Appl. No.: 10/831,756

(22) Filed: Apr. 26, 2004

Related U.S. Application Data
(63) Continuation of application No. 09/550,589, filed on Apr. 17, 2000.

(30) Foreign Application Priority Data
Apr. 16, 1999 (FR) 99 04818

(51) Int. Cl. A61K 7/06; A61K 7/11

(52) U.S. Cl. 424/70.12

(57) ABSTRACT

A cosmetic composition, in particular a make-up composition, containing at least one fatty acid ester or fatty alcohol ester, the carbonaceous chain of the fatty acid or alcohol being saturated and branched and comprising 24 to 28 carbon atoms, and at least one silicone compound. It also relates to the cosmetic applications of such a composition.

The present invention is directed to a cosmetic composition comprising an ester and a silicone compound. These compositions can constitute make-up compositions, such as powders, eyeshadows, foundations, concealers, lipsticks, make-up products for the body, mascaras or eyeliners, or alternatively, care compositions for the skin.

The use of silicone compounds in combination with hydrocarbonaceous compounds in cosmetic compositions, in particular make-up compositions, is known.

This is because silicone compounds make it possible, inter alia, to obtain, on the skin, a particularly homogeneous film which has good cosmetic properties. They also make it possible to improve the hold of foundation or lipstick compositions, for example, by virtue of their hydrophobic nature. In cosmetic powders, which generally comprise a particulate phase and a fatty phase as binder, they are incorporated into the binder in order to contribute softness.

Hydrocarbonaceous compounds are also used in cosmetic compositions. In the case of cosmetic powders, for example, the use is known, as binder, of a mixture of mineral and vegetable oils which are combined with esters, in order to obtain compositions which adhere well to the skin and are resistant to impacts.

Thus, it is advantageous to have available cosmetic compositions comprising both silicone compounds and hydrocarbonaceous compounds. However, it has been found that some silicone compounds are incompatible with some hydrocarbonaceous compounds generally used in cosmetic compositions, which limits the use thereof. In particular, numerous phase separation, release or syneresis phenomena are observed when these two types of compounds are brought into the presence of one another.

Various solutions have been provided in order to allow the preparation of a cosmetic composition comprising both silicone compounds and hydrocarbonaceous compounds. Mention may be made, for example, of the use of hydrocarbonaceous solvents or cosolvents, such as isoparaffins; however, their smell and their volatility are not always appreciated.

Furthermore, cosmetic compositions comprising these solubilizing agents contribute, when they are applied to the skin, a particularly unpleasant feeling of dryness, in particular in the case of lipsticks.

Furthermore, in the case of cosmetic powders, for example, the final composition must be sufficiently homogeneous and compact to be highly suitable for removal and, furthermore, to avoid fragmentation brought about in particular by impacts: in point of fact, this is rarely the case when the composition comprises a silicone compound.

Thus, while the use is already known of the combination of a silicone compound and of a hydrocarbonaceous compound, in particular an ester, in cosmetic compositions in general, it is still difficult to obtain a composition comprising these two types of compounds which is homogeneous and stable, which withstands being dropped, in the case of a compacted composition, and which also exhibits good cosmetic properties.

The aim of the present invention is to provide a homogeneous and resistant cosmetic composition which exhibits good cosmetic properties and which comprises at least one silicone compound in combination with an ester.

The Applicants have found, unexpectedly, that, by combining an ester, namely a fatty acid ester or fatty alcohol ester, the carbonaceous chain of the fatty acid or alcohol being saturated and branched and comprising 24 to 28 carbon atoms, with a silicone compound, it is possible to obtain a cosmetic composition which is not only very homogeneous on a lasting basis but also exhibits excellent cosmetic properties and excellent cohesion, in particular when the composition is in the compacted form.

An embodiment of the present invention is thus a cosmetic composition, in particular a make-up composition, characterized in that it comprises i) at least one fatty acid ester or fatty alcohol ester, the carbonaceous chain of the fatty acid or alcohol being saturated and branched and comprising 24 to 28 carbon atoms, and ii) at least one silicone compound.

The compositions according to the invention are homogeneous: thus, in general, a homogeneous distribution of the constituents is observed. In the case of solid/liquid mixtures, such as wax-in-oil mixtures, the formation is observed of a homogeneous and clear mixture under warm conditions and the formation is observed of a homogeneous dispersion of the wax in the oil during cooling. In the case of cosmetic sticks or powders, good dispersion of the pigments is observed and, in the case of compacted compositions or compositions in the form of sticks, good cohesion of the product is observed.

The compositions according to the invention remain homogeneous even after application to the skin and/or mucous membranes, this being the case for several hours.

The compositions according to the invention also exhibit excellent cosmetic properties. They are very soft, they adhere sufficiently but not too much to the skin and/or mucous membranes. They are readily applied and they provide a very pleasant feeling on application.

In particular, as regards the cosmetic powders, they are easy to compact, they disintegrate well and they exhibit good hardness. The compacted powders exhibit a notable ability to withstand being dropped and a very low percentage of

The compositions do not provide a feeling of dryness when they are applied to the skin and/or mucous membranes. They exhibit excellent hold. They also exhibit excellent dispersion of the pigments.

Another embodiment of the present invention is a process for the nontherapeutic treatment of the skin and/or mucous membranes, in particular a make-up process, which comprises applying, to the skin and/or mucous membranes, a composition as defined above.

Another embodiment of the present invention is the use of the combination of at least one silicone compound and of at least one fatty acid ester or fatty alcohol ester, the carbonaceous chain of the fatty acid or alcohol being saturated and branched and comprising 24 to 28 carbon atoms, in a compact cosmetic composition for the purpose of improving the impact strength of the composition.

Another embodiment of the present invention is the use of the combination of at least one silicone compound and of at least one fatty acid ester or fatty alcohol ester, the carbonaceous chain of the fatty acid or alcohol being saturated and branched and comprising 24 to 28 carbon atoms, in the preparation of a compact composition for the purpose of improving the impact strength of the composition.

The compositions according to the invention especially find a particularly advantageous application in the field of making up the skin, including the scalp, mucous membranes, semi-mucous membranes and superficial body growths. The term “mucous membrane” is understood to mean in particular the internal part of the lower eyelid. The term “semi-mucous membranes” is understood to mean more particularly the lips of the face. The term “superficial body growths” is understood to mean the eyelashes, eyebrows, hair and nails. Thus, the invention finds a very particular application in the field of products for caring for and/or making up the face and the skin, such as foundations, lipsticks, concealers, eyeshadows, powders for the face and body, or products for making up the body.

Other characteristics, aspects and advantages of the present invention will become apparent on reading the detailed description which will follow.

The compositions according to the invention comprise at least one fatty acid ester or fatty alcohol ester, the carbonaceous chain of the fatty acid or alcohol being saturated and branched and comprising 24 to 28 carbon atoms.

The word ester, according to the invention, means a monoester or a polyester. The term “polyester” is understood to mean, within the meaning of the present invention, a compound comprising more than one ester functional group, such as, for example, diesters, triesters, tetraesters and the like. The ester according to the invention is preferably chosen from polyesters. The ester according to the invention preferably comprises at least 2 branched (C₂₄ to C₂₈) chains. The word branched means at least one pendant hydrocarbonaceous chain comprising in particular from 1 to 14 carbon atoms.

The ester of the invention therefore comprises a saturated (C₂₄ to C₂.) fatty alcohol or fatty acid residue, in particular of the Guerbet fatty alcohol of following formula (a) or Guerbet fatty acid of following formula (b) type respectively:

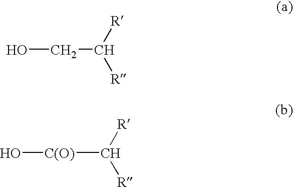

in which formulae R′ and R″ are saturated alkyl radicals, the sum of the carbon atoms of which ranges from 22 to 26. The R″ alkyl radical preferably comprises two carbon atoms less than the R′ alkyl radical.

Preferably, when the ester according to the invention derives from a Guerbet fatty alcohol, it is not derived from benzoic acid and it preferably derives from an aliphatic fatty acid.

The ester of the composition of the invention is preferably an oily ester which is liquid at room temperature (approximately 25° C.) exhibiting a high molecular weight, that is to say having a carbon number of greater than or equal to 50 and in particular of greater than or equal to 70. The advantage of a product which is liquid at room temperature in comparison with a product which is pasty or solid at room temperature lies in the greater number of its applications and its ease of use. Furthermore, the fact that this ester exhibits a high molecular weight makes it possible to obtain film-forming compositions which are persistent in water, which is widely desired for protection products, in particular sun protection products. This ester exhibits, inter alia, a refractive index of greater than 1.45 at 20° C. and an iodine number ≦4.

This ester, despite its high molecular weight, is neither greasy nor heavy nor sticky and confers, on the composition comprising it, notable comfort properties.

The ester according to the invention is advantageously an ester of a branched (C₂₄-C₂₈) fatty acid, such as 2-decyltetradecanoic acid, and more specifically an ester of a polyol, such as glycerol, which can be a mono-, di- or triglyceride. This ester is preferably a triglyceride of branched (C₂₄-C₂₈) fatty acids of the Guerbet type and in particular a triglyceride of a (C₂₄) fatty acid, such as 2-decyl-tetradecanoic acid.

Preferably, at least one of the branched chains of the ester comprises 24 carbon atoms.

More preferably, the ester according to the invention is chosen from triglycerides of a branched (C₂₄) fatty acid, pentaerythritol esters of a branched (C₂₄) fatty acid, esters of a branched (C₂₄) fatty alcohol and of diacids, and their mixtures.

A preferred triglyceride is, for example, glyceryl tri (2-decyltetradecanoate), sold under the reference DUB TGI 24 by the company Stearinerie Dubois. This ester exhibits a saponification number of 140 to 150, a refractive index >1.45, and in particular ranging from 1.454 to 1.459 at 20° C., an iodine number ≦4, a hydroxyl number ≦30 and an acid number ≦10. Its carbon number is 75.

Use may also be made of pentaerythritol esters of a (C₂₄) fatty acid, such as pentaerythrityl tetra(2-decyltetradecanoate) (with 101 carbon atoms), sold under the reference DUB PTI 24 by the company Stearinerie Dubois.

When the alcohol combined with the branched (C₂₄ to C₂₈) fatty acid is a polyol, the esterification can be partial (and can relate to 1, 2, 3 or more OH groups, depending on the alcohol used) or can be total.

Mention may be made, as ester of the invention comprising a fatty alcohol residue with a saturated branched (C₂₄ to C₂₈) chain, of di(decyltetradecyl) dimerates (with 84 carbon atoms), such as that sold under the reference DUB DI 24D by the company Stearinerie Dubois, or decyltetradecyl neopentanoate (with 29 carbon atoms) or decyltetradecyl isostearate, sold by the company Condea under the reference ISOFOL ESTER 2482 (with 42 carbon atoms). Dimerates are esters resulting from diacids, the latter generally being obtained from an unsaturated (C₆ to C₂₄) acid, such as oleic acid, linoleic acid, linolenic acid and the like.

The ester according to the invention preferably does not exhibit surfactant properties.

The ester according to the invention can be present in an amount which can range from 0.1 to 99.9% by weight with respect to the total weight of the composition and preferably in an amount ranging from 1 to 99% by weight with respect to the total weight of the composition.

In the case of a composition in the form of a liquid product for the complexion or for the lips, the ester is preferably present in an amount ranging from 1 to 99% by weight with respect to the total weight of the composition. In the case of a composition in the form of a stick product for the complexion or for the lips, the ester is preferably present in an amount ranging from 1 to 85% by weight, preferably from 1 to 30% by weight, with respect to the total weight of the composition.

In the case of a composition in the form of a powder, the ester according to the invention preferably represents from 0.1 to 23% by weight with respect to the total weight of the composition.

The compositions according to the invention additionally comprise a silicone compound.

In everything which follows or which precedes, the term “silicone compound” is understood to denote, in accordance with what is generally accepted, all organosilicon polymers or oligomers with a linear or cyclic, branched or crosslinked structure, of variable molecular weight, obtained by polymerization and/or polycondensation of suitably functionalized silanes and essentially composed of a repetition of main units in which the silicon atoms are connected to one another via oxygen atoms (siloxane bond ≡Si—O—Si≡), optionally substituted hydrocarbonaceous radicals being directly bonded via carbon atom to the said silicon atoms. The commonest hydrocarbonaceous radicals are alkyl radicals, in particular (C₁-C₃₀) alkyl radicals and especially methyl radicals, fluoroalkyl radicals or aryl radicals, in particular the phenyl radical.

The silicone compound can be present in the composition in an amount which can range from 0.1 to 99.9% by weight, preferably from 1 to 99% by weight, with respect to the total weight of the composition.

The silicone compound can be chosen from silicone oils, which may or may not be volatile, silicone waxes, silicone gums, silicone resins, silicone surfactants and/or their mixtures.

The term “silicone oil” is understood to mean, within the meaning of the present invention, a silicone which is liquid at room temperature (25° C.) which preferably exhibits a viscosity at 25° C. ranging from 0.5 to 500,000 cSt (centistokes).

Mention may be made, as silicone oil which is suitable for the present invention, of the linear polydimethylsiloxanes (PDMSs) corresponding to the following general formula (I):

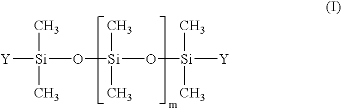

in which:

Y is —CH₃ or OH, and

m is an integer ranging from 0 to 2000.

Mention will in particular be made, among these, of the products sold under the name of AK by the company Wacker, SF by the company General Electric and ABIL by the company Goldschmidt, such as the product ABIL 10, and the products sold under the trade name DC 200 by Dow Corning.

Mention may also be made of:

poly(C₁-C₂₀)alkylsiloxanes and in particular those with end trimethylsilyl groups, preferably those with a viscosity of less than 0.06 m²/s (60,000 cSt), among which may be mentioned alkylmethylpolysiloxanes, such as cetyl dimethicone (CTFA name),

silicones modified by optionally fluorinated aromatic and/or aliphatic groups or by functional groups, such as hydroxyl, thiol and/or amine groups,

phenylated silicone oils.

Mention may also be made of volatile silicone oils, such as:

cyclic volatile silicones having from 3 to 8 silicon atoms and preferably from 4 to 6. They are, for example, cyclotetradimethylsiloxane, cyclopentadimethylsiloxane or cyclohexadimethylsiloxane,

cyclocopolymers of the dimethylsiloxane/methylalkylsiloxane type, such as SILICONE FZ3109, sold by the company Union Carbide, which is a dimethylsiloxane/methyloctylsiloxane

cyclocopolymer,

linear or volatile silicones having from 2 to 9 silicon atoms. They are, for example, hexamethyldisiloxane or a PDMS of low viscosity (1 cSt at 25° C.). Mention may also be made of alkyltrisiloxanes, such as hexylheptamethyltrisiloxane or octylheptamethyltrisiloxane.

Use will preferably be made of silicone oils with a viscosity at 25° C. varying from 5 cSt to 1500 cSt and more preferably from 10 cSt to 500 cSt.

Mention may more particularly be made, among poly(C₁-C₂₀)alkylsiloxanes, of cetyl dimethicone, sold under the trade name ABIL WAX 9801 by Goldschmidt, the viscosity of which at 25° C. is 15-25 cSt.

Mention may more particularly be made, among phenylated silicones, of those corresponding to the following formula (II):

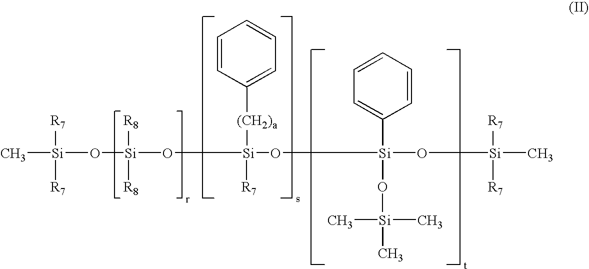

in which:

R₇ represents an alkyl radical comprising from 1 to 30 carbon atoms or an aryl radical, preferably a phenyl radical, or an aralkyl radical,

R₈ represents an alkyl radical comprising from 1 to 30 carbon atoms,

r is equal to 0 or 1,

s represents an integer between 0 and 100,

t represents an integer between 0 and 100,

a represents an integer between 0 and 10,

with the proviso that the sum r+s+t is between 1 and 100 and that, if s=t=O, then R₇ represents an aryl or aralkyl radical.

Mention may be made, among the phenylated silicones of formula (II), of the phenyl trimethylsiloxytrisiloxane with a viscosity of 20 cSt at 25° C. sold under the trade name DOW CORNING 556 FLUID by the company Dow Corning, the mixture of polydiphenyldimethylsiloxane and of phenyl trimethicone with a viscosity of 14 cSt at 25° C. sold under the trade name KF 56 by the company Shin-Etsu, or the diphenylmethyldimethyltrisiloxane with a viscosity of 50 cSt at 25° C. sold under the trade name DC-704 by the company Dow Corning.

The silicone oil can be present in the compositions of the invention in an amount which can range from 1 to 99% by weight with respect to the total weight of the composition.

In the case of a composition in the form of a liquid product for the complexion or for the lips, the silicone oil is preferably present in an amount ranging from 1 to 99% by weight with respect to the total weight of the composition. In the case of a composition in the form of a stick product for the complexion or for the lips, the silicone oil is preferably present in an amount ranging from 1 to 85% by weight, preferably from 1 to 30% by weight, with respect to the total weight of the composition.

In the case of a composition in the form of a powder, the silicone oil preferably represents from 0.1 to 23% by weight with respect to the total weight of the composition.

The term “silicone wax” is understood to mean, within the meaning of the present invention, a silicone compound which is solid or pasty at room temperature (25° C.), that is to say a compound exhibiting a melting point ranging from 25 to 130° C., preferably from 30 to 105° C.

A pasty compound will preferably have a melting point ranging from 25° C. to 50° C. and more preferably from 30° C. to 50° C.

A solid compound will preferably have a melting point ranging from 50° C. to 130° C. and more preferably from 50° C. to 105° C.

The silicone waxes which can be used according to the present invention are those generally used in cosmetics. Mention may more particularly be made among these, of the organopolysiloxanes corresponding to the following formula (III):

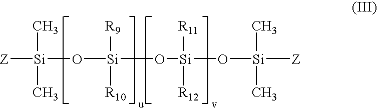

in which:

—R₉, R₁₁, and R₁₂ are, independently of one another, chosen from a (C₁-C₃₀) alkyl radical, a (C₁-C₃₀) alkoxy radical, and a (C₆-C₃₀) aryl radical,

R₁₀ is chosen from a (C₂-C₃₆) alkyl radical, a (C₂-C₃₆) alkoxy radical, a (C₂-C₃₆) alkyl radical, and a (C₂-C₃₆) alkoxy radical which is substituted by an ester group, it also being possible for R₁₀ to be a methyl radical when Z is a (C₂-C₃₀) alkyl radical, a (C₂-C₃₀) alkoxy radical, and a (C₂-C₃₀) ester,

Z is chosen from a (C₁-C₃₀) alkyl radical, a (C₁-C₃₀) alkoxy radical, a (C₆-C₃₀) aryl radical, and a (C₁-C₃₀) ester,

u is 0 or an integer between 1 and 100,

v is an integer between 1 and 100.

Mention may be made, for example, of the (C₂₄-C₂₈) alkyl dimethicone sold under the trade name ABIL WAX 9810 by the company Goldschmidt, the stearyl dimethicone sold under the trade name DC 2503 by the company Dow Corning, the dibehenoxy dimethicone sold under the trade name ABIL WAX 2440 by the company Goldschmidt, or the stearoxy dimethicone sold under the trade name VP 1622 by the company Wacker.

Use may also be made of the organosiloxanes obtained by the action of a natural wax, such as carnauba wax or beeswax, on a reactive silicone backbone.

The silicone wax can be present in an amount which can range from 0.1 to 50% by weight with respect to the total weight of the composition.

In the case of a composition in the form of a liquid product for the complexion or for the lips, the silicone wax is preferably present in an amount ranging from 0.1 to 5% by weight with respect to the total weight of the composition. In the case of a composition in the form of a stick product for the complexion or for the lips, the silicone wax is preferably present in an amount ranging from 0.1 to 50% by weight with respect to the total weight of the composition.

The term “gum” is understood to mean, within the meaning of the present invention, a compound with a viscosity at 25° C. ranging from 500,000 cSt to 5×10⁷ cSt which, in contrast to waxes, does not exhibit a melting point.

Mention may be made of the silicone gums corresponding to the following formula (IV):

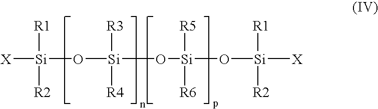

in which:

R1, R2, R5 and R6 are, independently of one another, an alkyl radical having 1 to 6 carbon atoms,

R3 and R4 are, together or separately, chosen from an alkyl radical having from 1 to 6 carbon atoms or an aryl radical, in particular a phenyl radical,

X is an alkyl radical having from 1 to 6 carbon atoms, a hydroxyl radical or a vinyl radical,

n and p being chosen so as to confer, on the silicone gum, a viscosity of greater than 500,000 cSt.

Generally, n and p can take values from 0 to 5000, preferably from 0 to 3000.

Mention may be made, as silicone gum which can be used according to the invention, of those for which:

the R1 to R6 and X substituents represent a methyl group, p=0 and n=2700, such as that sold under the name SE30 by the company General Electric,

the R1 to R6 and X substituents represent a methyl group, p=0 and n=2300, such as that sold under the name AK 500000 by the company Wacker,

the R1 to R6 substituents represent a methyl group, the X substituent represents a hydroxyl group, p=0 and n=2700, as a 13% solution in cyclopentasiloxane, such as that sold under the name Q2-1401 by the company Dow Corning,

the R1 to R6 substituents represent a methyl group, the X substituent represents a hydroxyl group, p=0 and n=2700, as a 13% solution in polydimethylsiloxane, such as that sold under the name Q2-1403 by the company Dow Corning,

the R1, R2, R5, R6 and X substituents represent a methyl group and the R3 and R4 substituents represent a phenyl group, such that the molecular weight of the compound is 600,000, such as that sold under the names 761 or Mirasil C-DPDM by the company Rhone-Poulenc.

The silicone gum can be present in an amount which can range from 0.1 to 20% by weight with respect to the total weight of the composition.

In the case of a composition in the form of a liquid product for the complexion or for the lips, the silicone gum is preferably present in an amount ranging from 0.1 to 0.20% by weight with respect to the total weight of the composition. In the case of a composition in the form of a stick product for the complexion or for the lips, the silicone gum is preferably present in an amount ranging from 0.1 to 10% by weight with respect to the total weight of the composition.

Silicone resins are products from the hydrolysis and polycondensation of mixtures of siloxanes of formulae R₃SiO_{1/2}, R₂SiO_{2/2}, RSiO_{3/2} and SiO_{4/2}, R representing an alkyl group having from 1 to 6 carbon atoms or a phenyl group. These resins can be dissolved in a solvent, such as a polydimethylsiloxane or polyphenylsiloxane oil or a cyclomethicone. These resins can be modified by an ester group, as in EP-A-602,905, the disclosure of which is hereby incorporated by reference, or by an alkyl group.

These silicone resins are known or can be prepared according to known methods.

Mention may be made, among commercial silicone resins which can be used, of, for example, those which are sold under the names DC 593 by Dow Corning or SS 4230 by General Electric.

The silicone resin can be present in an amount which can range from 0.1 to 90% by weight with respect to the total weight of the composition.

In the case of a composition in the form of a liquid product for the complexion or for the lips, the silicone resin is preferably present in an amount ranging from 0.1 to 70% by weight with respect to the total weight of the composition. In the case of a composition in the form of a stick product for the complexion or for the lips, the silicone resin is preferably present in an amount ranging from 0.1 to 50% by weight with respect to the total weight of the composition.

The silicone compound of the present invention can also be a silicone surfactant, in particular an oxyalkylenated silicone.

According to the invention, the term “oxyalkylenated silicone” denotes any silicone comprising at least one oxyalkylene-comp rising group of (—CₓH₂ₓO), type in which x is an integer ranging from 2 to 6 and a is an integer greater than or equal to 1.

Mention may be made, for example, of the products sold by the company Goldschmidt under the trade names ABIL WE 09, ABIL EM 90 or ABIL B8852, by the company Dow Corning under the names DC 3225 C, Q2-5220 or Q2-5200, or by the company Amerchol under the name SILSOFT BEAUTY AID SL.

The silicone surfactant can be present in an amount which can range from 0.1 to 40% by weight with respect to the total weight of the composition.

In the case of a composition in the form of a liquid product for the complexion or for the lips, the silicone surfactant is preferably present in an amount ranging from 0.1 to 40% by weight with respect to the total weight of the composition. In the case of a composition in the form of a stick product for the complexion or for the lips, the silicone surfactant is preferably present in an amount ranging from 0.1 to 30% by weight with respect to the total weight of the composition.

In addition to the above ester and the above silicone compound, the fatty phase of the composition according to the invention can comprise any other fatty substance, such as non-silicone oils and/or waxes and/or pasty fatty substances.

These fatty substances can be chosen from oils and/or waxes of mineral, animal, vegetable or synthetic origin (including fatty acid esters) or fluorinated oils.

Mention may be made, among the oils which can be used, of mink oil, turtle oil, soybean oil, grape seed oil, sesame oil, maize oil, rapeseed oil, sunflower oil, cottonseed oil, avocado oil, olive oil, castor oil, jojoba oil or groundnut oil; hydrocarbon oils, such as liquid paraffins, squalane, liquid petrolatum, polybutenes and hydrogenated polyisobutenes; fatty esters, such as isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate or lactate, di(2-ethylhexyl) succinate, diisostearyl malate, or glyceryl or diglyceryl triisostearate; perfluorinated oils; higher fatty acids, such as myristic acid, palmitic acid, stearic acid, behenic acid, oleic acid, linoleic acid, linolenic acid or isostearic acid; or higher fatty alcohols, such as cetanol, stearyl alcohol, oleyl alcohol or octyldodecanol.

Mention may be made, among the waxes which can be used, of beeswaxes, lanolin waxes and Chinese insect waxes; carnauba, candelilla or ouricury waxes, cork fibre waxes, sugar cane waxes, Japan waxes, hydrogenated jojoba waxes and hydrogenated oils, such as hydrogenated sunflower oil, hydrogenated castor oil, hydrogenated coconut oil and hydrogenated lanolin; paraffin waxes, microcrystalline waxes, montan waxes and ozokerites; polyethylene waxes, waxes obtained by the Fischer-Tropsch synthesis, or waxy copolymers and esters.

The composition of the invention can additionally comprise any additive conventionally used in the field under consideration (lacuna) antioxidants, essential oils, preservatives, neutralizing agents, non-silicone water-in-oil or oil-in-water surfactants, vitamins or antiwrinkle active principles.

The composition according to the invention can optionally comprise volatile oils other than silicone volatile oils.

The term “volatile oil” is understood to mean any compound capable of evaporating on contact with the skin. Use is preferably made of oils with a flash point sufficiently high to allow the use of these oils in formulation and sufficiently low to produce the desired evanescent effect. Oils with a flash point of the order of 40-100° C. are preferably employed.

These volatile compounds can be chosen in particular from hydrocarbonaceous oils, such as isoparaffins and in particular isododecane.

The composition according to the invention can also comprise a particulate phase which can comprise pulverulent compounds chosen from pigments and/or pearlescent agents and/or fillers and/or their mixtures commonly used in cosmetic compositions.

The term “pigments” should be understood as comprising white or colored and inorganic or organic particles which are insoluble in the medium and which are intended to color and/or opacify the composition.

The pigments can be present in a proportion of 0-40% by weight, preferably in a proportion of 0.1 to 30% by weight, with respect to the total weight of the composition and more preferably in a proportion of 1-20%. They can be white or colored, inorganic and/or organic, and conventional or nanometric in size. Mention may be made, among inorganic pigments and nanopigments, of titanium dioxides with a spherical shape or in the form of flakes, zirconium or cerium dioxides and zinc, iron or chromium oxides, titanium dioxide nanopigments or ferric blue. Mention may be made, among organic pigments, of carbon black and lakes, commonly employed to confer a make-up effect on the lips and skin, which are calcium, barium, aluminium or zirconium salts of acid dyes, such as haloacid, azo or anthraquinone dyes.

The pigments can in particular be coated with silicone compounds, such as PDMSs, and/or with polymers, in particular polyethylenes. Mention may thus be made of the SA pigments from Maprecos or the Pi pigments from Myoshi.

The term “fillers” should be understood as comprising colorless or white, inorganic or synthetic and lamellar or non-lamellar particles which are intended to give body or stiffness to the composition and/or softness, mattness and uniformity to the make-up.

The fillers can be present in the composition in a proportion of 0-99% by weight with respect to the total weight of the composition. In the case of a composition in the form of a powder, the fillers are preferably present in an amount ranging from 0.1 to 80% by weight with respect to the total weight of the composition. In the case of a composition in the form of a product for the complexion or for the lips, the fillers are preferably present in an amount ranging from 0.1 to 40% by weight, and more preferably in an amount ranging from 1 to 20% by weight, with respect to the total weight of the composition.

The fillers can be inorganic or synthetic and lamellar or non-lamellar. Mention may be made of talc, mica, silica, kaolin, powders formed of Nylon, of poly-β-alanine and of polyethylene, Teflon, lauroyllysine, starch, boron nitride, bismuth oxychloride, powders formed of tetrafluoroethylene polymers, poly(methyl methacrylate) powders, polyurethane powders, polystyrene powders, polyester powders, synthetic hollow microspheres, microsponges, silicone resin microbeads, zinc and titanium oxides, zirconium or cerium oxides, precipitated calcium carbonate, magnesium carbonate and hydrated magnesium carbonate, hydroxyapatite, hollow silica microspheres, glass or ceramic microcapsules, metal soaps derived from organic carboxylic acids having from 8 to 22 carbon atoms, preferably from 12 to 18 carbon atoms, such as zinc, magnesium or lithium stearate, zinc laurate or magnesium myristate, SiO₂/TiO₂/SiO₂, TiO₂/CeO₂/SiO₂ or TiO₂/ZnO/Talc compounds, or polyethylene terephthalate/polymethacrylate polymers in the form of flakes.

The term “pearlescent agents” should be understood as comprising iridescent particles which reflect the light.

The pearlescent agents can be present in the composition in a proportion of 0-60% by weight, preferably in a proportion of 0.1 to 40% by weight and more preferably in a proportion of 1-20% by weight with respect to the total weight of the composition. Mention may be made, among the pearlescent agents which can be envisaged, of natural mother-of-pearl, mica covered with titanium oxide, with iron oxide, with natural pigment or with bismuth oxychloride, and colored titanium oxide-coated mica.

In particular, by virtue of the combination according to the invention, it is possible to prepare compact powders comprising a high level of pearlescent agent.

The compositions of the invention additionally comprise a cosmetically, physiologically or dermatologically acceptable medium, that is to say a medium compatible with all keratinous substances, such as the skin, nails, hair, eyelashes and eyebrows, mucous membranes and semi-mucous membranes and any other cutaneous region of the body and face.

When the compositions of the invention are cosmetic powders, they preferably comprise from 77 to 99.9% by weight, with respect to the total weight of the composition, of particulate phase and 0.1 to 23% of fatty phase. They can be provided in the form of a powder, for example compacted, loose, pressed or cast. In the case of a loose powder, for example, the fatty phase can represent from 1 to 5% by weight with respect to the total weight of the composition. For a compacted powder, the content of fatty phase can represent from 1 to 20% by weight with respect to the total weight of the composition.

When the composition according to the invention is in the form of a powder, it is preferably in the form of a compact powder.

When the compositions of the invention are products for the complexion or for the lips, the fatty phase can represent from 5 to 100% by weight, preferably from 20 to 95% by weight, with respect to the total weight of the composition. These compositions can be provided in the form of an anhydrous gel.

The compositions according to the invention, in particular when they are provided in the form of products for the complexion or for the lips, can also comprise an aqueous phase.

The compositions of the invention can thus be provided in the form of an oil-in-water (O/W) emulsion, a water-in-oil (W/O) emulsion, a multiple emulsion or a multiphase solution.

The compositions of the invention can also be provided in the form of a vesicular dispersion, for example in the form of an oily phase dispersed in an aqueous phase and stabilized by liposomes.

When the composition according to the invention comprises an aqueous phase, the latter can be present in an amount ranging from 0.1 to 95% by weight with respect to the total weight of the composition. The aqueous phase can comprise water, a floral water, such as cornflower water, and/or a mineral water, such as water from Vittel, water from Lucas or water from La Roche-Posay, and/or a thermal water.

The aqueous phase can also additionally comprise water-soluble dyes chosen from the dyes usual in the field under consideration, such as the disodium salt of ponceau, the disodium salt of alizarin green, quinoline yellow, the trisodium salt of amaranth, the disodium salt of tartrazine, the monosodium-salt of rhodamine, the disodium salt of fuchsine, or xanthophyll.

The composition can moreover comprise any additional compound conventionally used in the cosmetics field, such as antioxidants, fragrances, essential oils, preservatives, lipophilic or hydrophilic cosmetic or pharmaceutical active principles, moisturizing agents, vitamins, essential fatty acids, sphingolipids, self-tanning compounds, such as DHA, or sunscreen agents.

These additional compounds can be present in the composition in a proportion of 0.1-20% by weight with respect to the total weight of the composition. Depending on their nature, they are present in the aqueous phase or in the fatty phase of the composition.

Of course, a person skilled in the art will take care to choose this or these optional additional compounds and/or their amounts so that the advantageous properties of the composition according to the invention are not, or not substantially, detrimentally affected by the envisaged addition.

The compositions according to the invention can be prepared according to conventional methods for the preparation of loose or compacted powders, W/O or O/W emulsions, or anhydrous gels, in the form of fluids, of creams, of pasty compositions or of sticks, these methods being well known to a person skilled in the art.

The invention is illustrated in more detail in the following examples.

In the following examples, the amounts are given as percentage by weight with respect to the total weight of the composition.

### EXAMPLE 1

### Comparative

The following compositions A (according to the invention) and B and C (comparative) were prepared:

| Talc | q.s. for 100% |
|---|---|
| Mica | 20% |
| BiOCl | 10% |
| TiO₂ | 2% |
| Metal salt | 3% |
| Pigments | 15% |
| Nylon | 20% |
| Binder | 5.5% |
| with: | |
| | Composition of the binder |
| | (as % by weight with respect to the total |
| Composition | weight of the binder) |
| Composition A (invention) | 50% glyceryl tri(2-decyltetradecanoate) |
| | 50% polydimethylsiloxane 10 cst |
| Composition B (comparative) | 50% glyceryl triisostearate |
| | 50% polydimethylsiloxane 10 cst |
| Composition C (comparative) | 68% polydimethylsiloxane 10 cSt |
| | 13.2% polydimethylsiloxane 100 cSt |
| | 6.5% trimethylsiloxysilicate |
| | 12.3% polymethylcetyldimethyl-siloxane |
| | 15-25 cSt |

These compositions were prepared in the following way: the pulverulent compounds were first mixed. The binder was added and mixing was again carried out. The combined mixture was sieved and then compacted in a dish at a compacting pressure of 80 bar.

Compact cosmetic powders were thus obtained which were, for example, eyeshadows.

The comparative compositions B and C each comprised a binder according to the prior art. The binder present in B comprised a silicone compound and glyceryl triisostearate, which is an ester with a chemical structure similar to glyceryl tri(2-decyltetradecanoate). The binder present in C was a mixture of silicone compounds not comprising a hydrocarbonaceous compound.

The respective impact strengths and hardnesses of the compositions A, B and C were compared.

The hardness of each composition was measured using a Zwick durometer graduated from 0 to 100 Shore A units. The needle of the durometer was made to penetrate close to the center of the dish in which the product to be measured was found and the hardness was read on the graduated dial.

Drop tests were carried out on each of the three compositions according to the following protocol: weighing was carried out on each sample compacted beforehand in a dish. Each sample was then dropped 10 times from a height of 20 cm vertically onto a sandstone slab. Each sample was again weighed and the percentage of product lost was calculated with respect to the weight of the initial product.

The results were collated in the table below:

| | % of loss of product after | Hardness |
|---|---|---|
| Composition | dropping | (° Shore) |
| Composition A (invention) | 6% | 55 |
| Composition B (comparative) | 14.3% | 58 |
| Composition C (comparative) | 28% | 55 |

Thus, for an equivalent hardness, the composition according to the invention, which comprises the “ester+polydimethylsiloxane” combination in accordance with the present invention, exhibited better results in the drop test than a composition comprising an “ester+polydimethylsiloxane” combination not in accordance with the present invention or than a composition comprising only a mixture of silicone compounds. The composition A according to the invention therefore exhibited better cohesion than the compositions of the prior art.

### EXAMPLE 2

### Comparative

Compositions D and E were prepared which correspond to the compositions according to Example 1 but in which the binder is:

composition D: glyceryl tri(2-decyltetradecanoate) alone,

composition E: polydimethylsiloxane alone.

The compositions A, B, C, D and E were respectively applied to the two eyelids of five people. The following criteria were subsequently evaluated:

the adhesion

the softness

the ease of application

the homogeneity

the overall assessment

It emerged from this that the composition A which comprised the combination according to the invention was better overall, as regards the above-mentioned cosmetic qualities, than the compositions B, C, D and E.

### EXAMPLE 3

The following composition F according to the invention and composition G (comparative) were prepared:

| Talc | q.s. for 100% |
|---|---|
| Mica | 10% |
| BiOCl | 3% |
| TiO₂ | 3% |
| Metal salt | 2% |
| Pigments | 4% |
| Nylon | 10% |
| Pearlescent agents | 50% |
| Binder | 18% |
| with: | |
| | Composition of the binder |
| | (as % by weight with respect to the total |
| Composition | weight of the binder) |
| Composition F (invention) | 50% glyceryl tri(2-decyltetradecanoate) |
| | 50% cetyl dimethicone 15-25 cSt |
| Composition G (comparative) | 100% glyceryl tri(2-decyltetradecanoate) |

These compositions were prepared according to the preparation method of Example 1 but were compacted at a compacting pressure of 130 bar. Despite the presence of 50% of pearlescent agents, it was possible to compact them.

The composition F exhibited good cohesion.

Furthermore, the cosmetic qualities of these two compositions were compared as in Example 2. It emerged from them that, for 75% of the people questioned, the composition F according to the invention was softer, easier to apply, more homogeneous and more pleasant overall than the composition G.

### EXAMPLE 4

The following lipstick in stick form was prepared:

| | |
|---|---|
| Polyethylene wax, sold under the | 11% |
| trade name PERFORMALENE 500 by | |
| the company New Phase Technologies | |
| Phase A | |
| Phenyl trimethicone, sold under the | 35.10% |
| trade name DC 556 by Dow Corning | |
| Glyceryl tri(2-decyltetradecanoate), | 35.11% |
| sold under the trade name DUB TGI 24 | |
| by the company Stéarinerie Dubois | |
| Liquid lanolin | 10.13% |
| Phase B | |
| FD & C Yellow No. 6, Al lake | 3.30% |
| Black iron oxide | 0.06% |
| DC Red No. 21, Al lake | 0.60% |
| Titanium dioxide | 1.80% |
| DC Red No. 7, Ca lake | 2.90% |

Procedure

The constituents of the phase A were mixed with magnetic stirring at 60° C. After homogenizing, the pigments (phase B) were added and the combined mixture was milled using a triple roll mill.

The wax was subsequently added and the mixture was heated to 98° C.

After homogenizing using a magnetic stirrer, the composition was cast at 98° C. in a suitable mould in order to obtain a stick of lipstick.

The stick of lipstick thus prepared was particularly homogeneous.

Furthermore, this composition was characterized by great softness and good gloss on application.

### EXAMPLE 5

The following stick of lipstick H was prepared:

| Composition H | |
|---|---|
| Polyethylene wax, sold under the | 12% |
| trade name PERFORMALENE 500 by | |
| the company New Phase Technologies | |
| Nylon powder | 5.00% |
| Cyclopentadimethylsiloxane | 34.34% |
| Phase A | |
| Silicone wax, sold under the name | 5.00% |
| DC 2503 by the company Dow Corning | |
| Phenyl trimethicone, sold under the | 17.50% |
| name DC 556 by the company Dow Corning | |
| Glyceryl tri(2-decyltetradecanoate), | 17.50% |
| sold under the trade name DUB TGI 24 | |
| by the company Stéarinerie Dubois | |
| Phase B | |
| FD & C Yellow No. 6, Al lake | 3.30% |
| Black iron oxide | 0.06% |
| DC Red No. 21, Al lake | 0.60% |
| Titanium dioxide | 1.80% |
| DC Red No. 7, Ca lake | 2.90% |

Procedure

The constituents of the phase A were mixed with magnetic stirring at 60° C. After homogenizing, the pigments (phase B) were added and the combined mixture was milled using a triple roll mill.

The polyethylene wax was subsequently added and the mixture was heated to 98° C.

After homogenizing using a magnetic stirrer, Nylon powder and the cyclopentadimethylsiloxane were added.

After homogenizing, the composition was cast at 98° C. in a suitable mould in order to obtain a stick of lipstick.

Composition J, which is identical to the composition H except for the difference that the 17.5% of glyceryl tri(2-decyltetradecanoate) was replaced by 17.5% of phenyl trimethicone, was subsequently prepared.

The compositions H and J were subsequently compared with respect to their cosmetic properties. The following criteria were subsequently evaluated:

smoothness on application,

gloss on application,

slip on application,

coverage,

and were judged to be better for the stick H according to the invention.

In particular, the stick H exhibited excellent dispersion of the pigments in the composition and thus good homogeneity.

1-77. (Canceled)

78. A compacted cosmetic powder composition comprising:
at least one polyester comprising at least one fatty acid ester or fatty alcohol ester, comprising at least two saturated and branched carbonaceous chains of 24 to 28 carbon atoms,
at least one silicone compound, and
a particulate phase comprising at least one pulverulent compound,
wherein the compacted cosmetic powder composition is homogeneous.

79. The composition according to claim 78, wherein the composition is a make-up composition.

80. The composition according to claim 78, wherein the at least one polyester has a carbon number of greater than or equal to 50.

81. The composition according to claim 80, wherein the at least one polyester has a carbon number of greater than or equal to 70.

82. The composition according to claim 80, wherein at least one of the at least two saturated and branched chains comprises 24 carbon atoms.

83. The composition according to claim 78, wherein the at least one fatty acid ester or fatty alcohol ester is chosen from triglycerides of a branched C₂₄ fatty acid, pentaerythritol esters of a branched C₂₄ fatty acid, esters of a branched C₂₄ fatty alcohol and of diacids, and their mixtures.

84. The composition according to claim 78, wherein the at least one polyester has a refractive index of greater than 1.45 at 20° C.

85. The composition according to claim 78, wherein the at least one polyester has an iodine number of less than or equal to 4.

86. The composition according to claim 78, wherein the at least one polyester is glyceryl tri(2-decyltetradecanoate).

87. The composition according to claim 78, wherein the at least one polyester is present in the composition in an amount ranging from 0.1 to 99.9% by weight with respect to the total weight of the composition.

88. The composition according to claim 87, wherein the at least one polyester is present in the composition in an amount ranging from 1 to 99% by weight with respect to the total weight of the composition.

89. The composition according to claim 78, wherein the at least one silicone compound is chosen from a silicone oil, which may or may not be volatile, a silicone wax, a silicone gum, a silicone resin, a silicone surfactant and/or their mixtures.

90. The composition according to claim 78, wherein the at least one silicone compound is present in the composition in an amount ranging from 0.1 to 99.9% by weight with respect to the total weight of the composition.

91. The composition according to claim 90, wherein the at least one silicone compound is present in the composition in an amount ranging from 1 to 99% by weight with respect to the total weight of the composition.

92. The composition according to claim 78, wherein the at least one silicone compound is chosen from a silicone oil having a viscosity at 25° C. ranging from 5 cSt to 1500 cSt.

93. The composition according to claim 92, wherein the at least one silicone compound is chosen from a silicone oil having a viscosity at 25° C. ranging from 10 cSt to 500 cSt.

94. The composition according to claim 89, wherein the silicone oil is chosen from a phenylated silicone oil corresponding to the following formula (II): 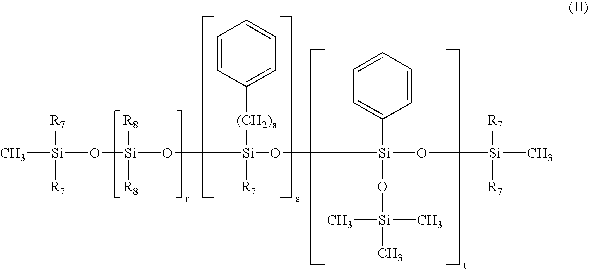
wherein:
R₇ is chosen from an alkyl radical comprising from 1 to 30 carbon atoms, an aralkyl radical, and an aryl radical,
R₈ is an alkyl radical comprising from 1 to 30 carbon atoms,
r is equal to 0 or 1,
s is an integer ranging from 0 to 100,
t is an integer ranging from 0 to 100,
a is an integer ranging from 0 to 10,
with the proviso that the sum r+s+t ranges from 1 and 100 and that, if s=t=0, then R₇ represents an aryl or aralkyl radical.

95. The composition according to claim 94, wherein R₇ is a phenyl radical or an aralkyl radical.

96. The composition according to claim 89, wherein the volatile silicone oil is chosen from a cyclic volatile silicone having from 3 to 8 silicon atoms, a dimethylsiloxane/methylalkylsiloxane cyclocopolymer, and a linear volatile silicone having from 2 to 9 silicon atoms.

97. The composition according to claim 89, wherein the silicone oil is present in the composition in an amount ranging from 1 to 99% by weight with respect to the total weight of the composition.

98. The composition according to claim 89, wherein the at least one silicone compound is chosen from a silicone wax corresponding to the following formula (III): 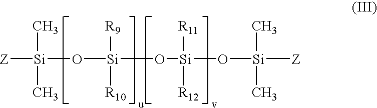
in which:
R₉, R₁₁ and R₁₂ are, independently of one another, chosen from a (C₁-C₃₀) alkyl radical, a (C₁-C₃₀) alkoxy radical, and a (C₆-C₃₀) aryl radical,
R₁₀ is chosen from a (C₂-C₃₆) alkyl radical, a (C₂-C₃₆) alkoxy radical, a (C₂-C₃₆) alkyl radical, and a (C₂-C₃₆) alkoxy radical which is substituted by an ester group, it also being possible for R₁₀ to be a methyl radical when Z is chosen from a (C₂-C₃₀) alkyl radical, a (C₂-C₃₀) alkoxy radical, and a (C₂-C₃₀) ester,
Z is chosen from a (C₁-C₃₀) alkyl radical, a (C₁-C₃₀) alkoxy radical, a (C₆-C₃₀) aryl radical, and a (C₁-C₃₀) ester,
u is 0 or an integer ranging from 1 to 100, and
v is an integer ranging from 1 to 100.

99. The composition according to claim 98, wherein the silicone wax is chosen from (C₂₄-C₂₈) alkyl dimethicone, stearyl dimethicone, dibehenoxy dimethicone, and stearoxy dimethicone.

100. The composition according to claim 99, wherein the silicone wax is present in the composition in an amount ranging from 0.1 to 50% by weight with respect to the total weight of the composition.

101. The composition according to claim 89, wherein the at least one silicone compound is chosen from a silicone gum corresponding to the following formula (IV): 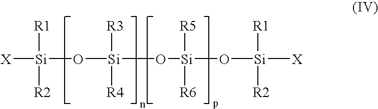
in which:
R1, R2, R5 and R6 are, together or separately, an alkyl radical having 1 to 6 carbon atoms,
R3 and R4 are, together or separately, an alkyl radical having from 1 to 6 carbon atoms or an aryl radical
X is chosen from an alkyl radical having from 1 to 6 carbon atoms, a hydroxyl radical, and a vinyl radical,
n and p being chosen so as to confer, on the silicone gum, a viscosity of greater than 500,000 cSt.

102. The composition according to claim 101, wherein R3 and R4 are, together or separately, a phenyl radical.

103. The composition according to claim 101, wherein the silicone gum is present in the composition in an amount ranging from 0.1 to 20% by weight with respect to the total weight of the composition.

104. The composition according to claim 89, wherein the silicone resin is chosen from a mixture of siloxanes of formulae R₃SiO_{1/2}, R₂SiO_{2/2}, RSiO_{3/2} and SiO_{4/2}, wherein R is an alkyl group having from 1 to 6 carbon atoms or a phenyl group.

105. The composition according to claim 104, wherein the silicone resin is present in the composition in an amount ranging from 0.1 to 90% by weight with respect to the total weight of the composition.

106. The composition according to claim 89, wherein the silicone surfactant is chosen from a silicone comprising at least one oxyalkylene-comprising group of the formula (—CₓH₂ₓO)ₐ in which x is an integer ranging from 2 to 6 and a is an integer greater than or equal to 1.

107. The composition according to claim 106, wherein the silicone surfactant is present in the composition in an amount ranging from 0.1 to 40% by weight with respect to the total weight of the composition.

108. The composition according to claim 78, further comprising at least one oil chosen from mink oil, turtle oil, soybean oil, grape seed oil, sesame oil, maize oil, rapeseed oil, sunflower oil, cottonseed oil, avocado oil, olive oil, castor oil, jojoba oil, groundnut oil, hydrocarbon oils, fatty esters, perfluorinated oils, fatty acids, glyceryl, and fatty alcohols.

109. The composition according to claim 108, wherein the hydrocarbon oils are chosen from liquid paraffins, squalane, liquid petrolatum, polybutenes and hydrogenated polyisobutenes.

110. The composition according to claim 108, wherein the fatty esters are chosen from isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, 2-octyldodecyl lactate, di(2-ethylhexyl) succinate, diisostearyl malate, and diglyceryl triisostearate.

111. The composition according to claim 108, wherein the higher fatty acids are chosen from myristic acid, palmitic acid, stearic acid, behenic acid, oleic acid, linoleic acid, linolenic acid, and isostearic acid.

112. The composition according to claim 108, wherein the higher fatty alcohols are chosen from cetanol, stearyl alcohol, oleyl alcohol, and octyldodecanol.

113. The composition according to claim 78, further comprising at least one wax chosen from beeswaxes, lanolin waxes, Chinese insect waxes, carnauba, candelilla waxes, ouricury waxes, cork fibre waxes, sugar cane waxes, Japan waxes, hydrogenated jojoba waxes, hydrogenated oils, paraffin waxes, microcrystalline waxes, montan waxes, ozokerites, polyethylene waxes, waxes obtained by the Fischer-Tropsch synthesis, waxy copolymers and esters.

114. The composition according to claim 113, wherein the hydrogenated oils are chosen from hydrogenated sunflower oil, hydrogenated castor oil, hydrogenated coconut oil, and hydrogenated lanolin.

115. The composition according to claim 78, wherein the at least one pulverulent compound is chosen from pigments, pearlescent agents, fillers, and their mixtures.

116. The composition according to claim 115, wherein the pigments are chosen from titanium dioxides, with a spherical shape or in the form of flakes, zirconium dioxides, cerium dioxides, zinc oxides, iron oxides, chromium oxides, titanium dioxide nanopigments, ferric blue, carbon black, calcium, barium, aluminium salts of acid dyes, zirconium salts of acid dyes, pigments coated with silicone compounds, and pigments coated with polymers.

117. The composition according to claim 116, wherein the zirconium salts of acid dyes are chosen from zirconium salts of haloacid, azodyes, and anthraquinone dyes.

118. The composition according to claim 116, wherein the pigments coated with silicone compounds are pigments coated with a polydimethylsiloxane.

119. The composition according to claim 116, wherein the pigments are present in the composition in an amount ranging from greater than 0 to 40% by weight with respect to the total weight of the composition.

120. The composition according to claim 119, wherein the pigments are present in the composition in an amount ranging from 0.1 to 30% by weight with respect to the total weight of the composition.

121. The composition according to claim 115, wherein the fillers are chosen from talc, mica, silica, kaolin, powders formed of Nylon, powders formed of poly-β-alanine, powders formed of polyethylene, Teflon, lauroyllysine, starch, boron nitride, bismuth oxychloride, powders formed of tetrafluoroethylene polymers, poly(methyl methacrylate) powders, polyurethane powders, polystyrene powders, polyester powders, synthetic hollow microspheres, microsponges, silicone resin microbeads, zinc oxides, titanium oxides, zirconium oxides, cerium oxides, precipitated calcium carbonate, magnesium carbonate, hydrated magnesium carbonate, hydroxyapatite, hollow silica microspheres, glass microcapsules, ceramic microcapsules, metal soaps derived from organic carboxylic acids having from 8 to 22 carbon atoms, SiO₂/TiO₂/SiO₂ compounds, TiO₂/CeO₂/SiO₂ compounds, TiO₂/ZnO/Talc compounds, and polyethylene terephthalate/polymethacrylate polymers in the form of flakes.

122. The composition according to claim 121, wherein the metal soaps derived from organic carboxylic acids have from 12 to 18 carbon atoms.

123. The composition according to claim 122, wherein the metal soaps are chosen from zinc stearate, magnesium stearate, lithium stearate, zinc laurate, and magnesium myristate.

124. The composition according to claim 121, wherein the fillers are present in the composition in an amount ranging from greater than 0 to 99% by weight with respect to the total weight of the composition.

125. The composition according to claim 115, wherein the pearlescent agents are chosen from natural mother-of-pearl, mica covered with titanium oxide, mica covered with iron oxide, mica covered with natural pigment, mica covered with bismuth oxychloride, and colored titanium oxide-coated mica.

126. The composition according to claim 125, wherein the pearlescent agents are present in the composition in an amount ranging from greater than 0 to 60% by weight with respect to the total weight of the composition.

127. The composition according to claim 126, wherein the pearlescent agents are present in the composition in an amount ranging 0.1 to 40% by weight with respect to the total weight of the composition.

128. The composition according to claim 78, wherein the composition additionally comprises an aqueous phase in an amount ranging from 0.1 to 95% by weight with respect to the total weight of the composition.

129. The composition according to claim 115, wherein the fillers are present in the composition in an amount ranging from 0.1 to 40% by weight with respect to the total weight of the product.

130. The composition according to claim 115, wherein the fillers are present in the composition in an amount ranging from 1 to 20% by weight with respect to the total weight of the product.

131. A compacted cosmetic powder composition for the complexion comprising:
at least one polyester comprising at least one fatty acid ester or fatty alcohol ester comprising at least two saturated and branched carbonaceous chains of 24 to 28 carbon atoms,
at least one silicone compound, and
a particulate phase comprising at least one pulverulent compound,
wherein the composition is homogeneous, and
wherein the composition is applied to skin.

132. The composition according to claim 131, wherein the composition additionally comprises at least one wax, wherein the at least one wax is present in the composition in an amount ranging from 0.1 to 5% by weight with respect to the total weight of the composition.

133. The composition according to claim 131, wherein the at least one silicone compound is a silicone resin, wherein the silicone resin is present in the composition in an amount ranging from 0.1 to 70% by weight with respect to the total weight of the product.

134. A compacted cosmetic powder comprising a composition comprising:
at least one fatty acid ester or fatty alcohol ester, the carbonaceous chain of the fatty acid or alcohol being saturated and branched, and comprising from 24 to 28 carbon atoms, and
at least one silicone compound,
wherein the cosmetic powder is homogeneous.

135. The powder according to claim 134, wherein the at least one fatty acid ester or fatty alcohol ester is present in the powder in an amount ranging from 0.1 to 23% by weight with respect to the total weight of the powder.

136. The powder according to claim 134, wherein the at least one silicone compound comprises at least one silicone oil, and wherein the at least one silicone oil is present in the powder in an amount ranging from 0.1 to 23% by weight with respect to the total weight of the powder.

137. The powder according to claim 134, wherein the powder additionally comprises at least one filler, wherein the at least one filler is present in the powder in an amount ranging from 0.1 to 80% by weight with respect to the total weight of the powder.

138. The powder according to claim 134, wherein composition additionally comprises a particulate phase, and wherein the particulate phase is present in the powder in an amount ranging from 77 to 99.9% by weight with respect to the total weight of the powder.

139. A process for the nontherapeutic treatment of the skin and/or mucous membranes comprising:
applying, to the skin and/or mucous membranes, a compacted cosmetic powder composition comprising at least one polyester comprising at least one fatty acid ester or fatty alcohol ester comprising at least two saturated and branched carbonaceous chains of 24 to 28 carbon atoms, at least one silicone compound, and a particulate phase comprising at least one pulverulent compound,
wherein the composition is homogeneous.

140. The process according to claim 139, wherein the process is a make-up process.

141. A method of improving the impact strength of a compacted cosmetic powder composition comprising combining at least one silicone compound, at least one polyester chosen from at least one fatty acid ester or fatty alcohol ester comprising at least two saturated and branched carbonaceous chains of 24 to 28 carbon atoms, and a particulate phase comprising at least one pulverulent compound,
wherein the compacted cosmetic power composition is homogeneous, and
wherein the combination provides improved impact strength.

142. A method of preparing a compacted cosmetic powder composition comprising combining at least one silicone compound, at least one polyester chosen from at least one fatty acid ester or fatty alcohol ester comprising at least two saturated and branched carbonaceous chains of 24 to 28 carbon atoms, and a particulate phase comprising at least one pulverulent compound,
wherein the compacted cosmetic powder composition is homogeneous, and
wherein the combination provides a compacted cosmetic composition with improved impact strength.

143. The composition according to claim 131, wherein the at least one pulverulent compound is chosen from pigments, pearlescent agents, fillers, and their mixtures.

144. The process according to claim 139, wherein the at least one pulverulent compound is chosen from pigments, pearlescent agents, fillers, and their mixtures.

145. The method according to claim 141, wherein at least one pulverulent compound is chosen from pigments, pearlescent agents, fillers, and their mixtures.

146. The method according to claim 142, wherein at least one pulverulent compound is chosen from pigments, pearlescent agents, fillers, and their mixtures.
